(19) United States
(12) Patent Application Publication
(10) Pub. No.: US 2023/0285337 A1
(43) Pub. Date: Sep. 14, 2023

(54) LONG ACTING ANTICANCER COMPOSITIONS

(71) Applicant: INTRAGEL THERAPEUTICS LTD., Nazereth (IL)

(72) Inventor: Abraham Jakob DOMB, Jerusalem (IL)

(21) Appl. No.: 18/040,443

(22) PCT Filed: Aug. 5, 2021
(86) PCT No.: PCT/IL2021/050952

§ 371 Date: Feb. 3, 2023

Related U.S. Application Data
(60) Provisional application No. 63/062,557, filed on Aug. 7, 2020.

(51) Int. Cl. A61K 31/194; A61K 33/243; A61K 31/201; A61K 31/555; A61P 35/00; A61K 9/00

(52) U.S. Cl. CPC A61K 31/194; A61K 9/0019; A61K 31/201; A61K 31/555; A61K 33/243; A61P 35/00

(57) ABSTRACT

The invention generally concerns stable anticancer formulations.

### FIELD OF THE INVENTION

The present invention relates to long acting anticancer compositions.

### BACKGROUND OF THE INVENTION

Cancer is a group of more than one-hundred complex diseases characterized by their ability to invade adjacent tissues and spread throughout the body. It is the second leading cause of death in the US, where more than nine million people are living with cancer. Solid cancers account for over 70% of the newly diagnosed cancer cases each year, or more than 1.75 million cases in the Western World.

Solid tumors are commonly treated as a local and systemic disease in which the solid tumor is surgically removed and/or irradiated or ablated by local heating or freezing. This may be followed by systemic chemotherapy. Standard chemotherapy is a systemic treatment administered through injections, intravenously, or orally with the intent to kill tumor cells that may spread locally or to new sites throughout the body.

In cancer patients, obstruction of the gastrointestinal tract and airway results with severe feeding and breathing difficulties. In many instances stent insertion may relief these symptoms. Maintenance of stent patency has an influential factor affecting the quality of life, but not without a high rate of complexity and risk. Patients are usually pretreated heavily at the time of obstruction, but poor nutritional status cannot tolerate systemic chemotherapy at standard dosage. Local delivery of an anticancer drug in high concentration can prevent re-growth, but results in stent occlusion.

Although solid tumors are treated as a local disease (surgery, irradiation, ablation), there is no localized chemotherapy that delivers high doses of anticancer agents at the tumor site for an extended time period with low systemic distribution. Localized doses reaching 100 times higher than the maximal concentration achieved by systemic chemotherapy (without risk to the patient) can serve as an alternative or complement to surgery, irradiation, and systemic chemotherapy. As most solid tumors are accessible via current biopsy techniques, localized delivery systems releasing effective doses of one or more anticancer agents may be used for weeks either for pre-operative tumor size reduction or for post-operative, complementary eradication of remaining tumor cells in the tumor bed, and treatment of non-operable solid tumors.

There have been attempts to develop clinical formulations to treat head and neck cancer. IntraDose® Injectable Gel is a collagen aqueous solution containing cisplatin and epinephrine for recurrent squamous cell carcinoma of the head and neck, and OncoGel PLA-PEG is an aqueous solution loaded with paclitaxel for esophageal cancer. These systems developed by MacroMed (Re-Gel) and Access Pharmaceuticals failed in clinical trials because of toxicity and lack of effectiveness due to the washout of the drug from the aqueous carriers shortly after injection into tissue. OncoGel™ (paclitaxel in Re-Gel, injectable solution of PEG-PLA copolymer in water at <20° C. that gels at body temperature) has been in clinical development for several years. When the formulation gels, the drug is leached out with water and only little remains for controlled release. In a clinical study OncoGel did not show any impact on the primary endpoint of overall tumor response in a Phase IIb study exploring its use as a neoadjuvant therapy to standard chemotherapy and radiation therapy before surgery in patients with oesophageal cancer. A follow-up study of the secondary outcome measure of patient survival has been discontinued, since there can be no anticipated impact.

Little has been done to develop safe and effective intratumoral extended focal delivery of anticancer agents for treating solid tumors. The key for a successful delivery system is the polymer carrier that should possess predictable and reproducible molecular weight, predictable and controlled polydispersity, viscosity and injectability -- if designed as injectable formulation and made from natural components that are naturally metabolized and eliminated from the body -- predictable and reproducible controlled release of the incorporated drug over a desired period that can be from one to eight weeks with minimal burst release to avoid toxic blood levels, no or tolerable toxicity at the injection site and the body, fully degradable to natural degradation products that are metabolized and eliminated from the body shortly after the drug has been depleted, simple drug incorporation with no or minimal use of heat, sheer forces, toxic solvents, aqueous media that may prematurely degrade the polymer carrier or affect the active agents, processes that do not require special equipment and storage stability of the polymer and polymer-drug formulation at refrigeration or room temperature for months.

Polyanhydrides have been investigated as carriers for the controlled delivery of several drugs due to their surface eroding properties. Polyanhydrides have inherent high reactivity toward water, which prompts rapid hydrolytic degradation. Due to the high rate of hydrolysis, polyanhydrides endure surface erosion rather than bulk degradation. Gliadel wafer, an approved polyanhydride copolymer of carboxyphenoxy propane and sebacic acid, is a bioresorbable medicinal implant that is used to deliver carmustine, an anticancer agent to cerebral tumor sites. Polyanhydride based particles have been widely studied in many formulations for effective drug delivery. Nevertheless, the number of polyanhydride products existing in the market is only one compared to dozens for polyester products. Even though polyanhydrides are easy and inexpensive to synthesize and scale up, they exhibit a short shelf-life under common storage conditions. Polyanhydrides are prone to hydrolytic degradation and depolymerization via anhydride interchange during storage, and may therefore be produced along with decomposition products. Hence, polyanhydrides need to be kept at freezing storage conditions that restrict their usage in drug delivery products. Accordingly, the usability of polyanhydride products in the medical fields (e.g. carriers of drugs) is less attractive. One such example of a stable polyanhydride is the poly(ester-anhydride) based on the ricinoleic acid and sebacic acid reported in [1-3].

### REFERENCES

US 10,774,176
US 2020/0101163
Domb et al., 2017, J of Controlled Release, 257, 156-162.

### SUMMARY OF THE INVENTION

This invention describes a unique biodegradable and biocompatible polymer based anticancer composition. The polymeric anticancer formulation may be injected or inserted into a tissue through a needle or trocar. It then gels on contact with body fluids to form a depot implant releasing the drug at the tumor site over a period of several weeks, in a controlled manner. The delivery system provides a high local concentration of an anticancer drug that destroys malignant cells that may have survived surgery, thus preventing re-growth and metastasis of solid tumors. The depot polymer implant provides extended release of the loaded drug for periods of weeks with minimal systemic drug distribution, thus providing a safer and more effective alternative to standard systemic chemotherapy.

The polymeric formulation of the invention is based on a polyanhydride exhibiting improved properties to those previously disclosed in the prior art. The polyanhydride is of the form —(SA—RA)n—, wherein SA is sebacic acid and RA is ricinoleic acid, and wherein n is an integer between 10 and 100, prepared by melt condensation of SA and RA with a mole equivalent or less of acetic anhydride per carboxylic acid group, and in the absence of a solvent, the polyanhydride being a narrow-polydispersed polymer. This polyanhydride is referred herein as the polymer of the invention or the carrier of the invention.

The absence of a solvent and the sequential addition of the various precursors allows for producing a final product that is well characterized and reproducible to meet regulatory requirements of the highest standards and which exhibits narrow polydispersity. The term “narrow polydispersity” or any lingual variation thereof, when made in reference to a polymer of the invention defines a collection of materials having substantially identical compositions (type of repeating groups and manner of repetition) and molecular weights. The narrow polydispersity of a polymer of the invention, defined by the ratio Mw/Mn (wherein Mw is the weight-average molecular weight and Mn is the number-average molecular weight) is below 2.5 or below 2. Putting it differently, the narrow disperse or narrow polydisperse polymer of the invention has a polydispersity value of no more than 2.5 or 2 (or a value between 2.5 and 1, or between 2 and 1).

Polymers of the invention also exhibit high reproducibility, namely a reproducibility in polymer molecular weight that is no more than 30% deviation from polymer average molecular weight.

The term “in absence of a solvent” herein refers to the property of the process of the invention as having no or a minute amount of solvent(s) that may be derived from impurities present with the precursor materials. Such impurities will not exceed 0.001%, 0.005%, 0.01%, 0.05% or 0.1% (w/w) of the total weight of the reaction materials used.

The polymer of the invention is prepared by a process comprising:
-reacting sebacic acid (SA) and ricinoleic acid (RA) under conditions permitting esterification of the SA (to obtain a mono ester of SA or a di-ester thereof or a mixture thereof); and
-transforming the esterified SA (mono or di- or mixture thereof) into the narrow-polydisperse polyanhydride.

The process of the invention permits for direct condensation in bulk (in the melt), without a pre-reaction to form a polymer or an oligomer of any of the material precursors used. In an exemplary process, sebacic acid (SA) (a dicarboxylic acid) was reacted with ricinoleic acid (RA) (a hydroxyl-alkanoic acid) at a 30:70 w/w ratio to form a mixture of SA-RA dimers and RA-SA-RA trimers with minimal or no RA or RA-RA ester molecules in the reaction product. The SA-RA and RA-SA-RA mixture (free of the precursor molecules and of the RA-RA molecules) is thereafter treated with no more than one molar equivalent of acetic anhydride per free carboxylic acid group (being typically 2 free carboxylic acid groups and thus no more than 2 molar equivalents) to acetylate the free ester and thereafter polymerize the acetylated segments into the narrow-dispersed polyanhydride having the repeating ...RA-SA-RA-SA...sequence. The process is depicted in FIG. 1.

Mixture of dimers and trimers of SA and RA can be used to form a heterogeneous polymer consisting anhydride bonds and ester bonds between SA and RA with minimal ester bonds between two RA units. On the other hand, formation of anhydride diads of the SA monomers along the polymer chain, may limit the storage stability of the polymer. Thus, in a process of the invention, the molar ratio between a SA and RA is typically equivalent or in favor of RA. In other words, the amount of the RA is preferably equal to or double (1:1 to 1:2 molar equivalent) that of SA. In some embodiments, the weight ratio SA:RA is 1:1, 1:1.1, 1:1.2, 1:1.3, 1:1.4, 1:1.5, 1:1.6, 1:1.7, 1:1.8, 1:1.9, 1:2, respectively.

In some embodiments, the molar ratio between the SA:RA ranges between 1:1 and 1:2, respectively to avoid ester bond formation between RA units, so that the polymer comprises anhydride bonds and ester bonds only between SA and RA.

In some embodiments, the weight ratio is 30:70, 35:65 or 25:75 for SA and RA building blocks, respectively.

An excess amount of the RA permits mono- and di-esterification of the SA (with some amount of a mono esterified form), and avoids formation of ester dimers of the RA. The SA-RA and SA-RA-SA mixture (herein a “dimer-trimer mixture”) is obtained by heating a mixture of SA and RA, in the indicated ratios, at a temperature above 80° C. In some embodiments, the temperature is between 80 and 200, between 100 and 190, between 100 and 180, between 100 and 170, between 100 and 160, between 100 and 150, between 100 and 140, between 100 and 130, or between 100 and 120° C.

The condensation of the two components involves direct ester condensation to provide the dimer-trimer dicarboxylic acid oligomer mixture. The dimer-trimers oligomers are polymerized into a polyanhydride by activation of the carboxylic acid ends with acetic anhydride. The amount of the acetic anhydride used is not greater than one molar equivalent of acetic anhydride per every free carboxylic acid group in the oligomers. The dimer SA-RA has two free carboxylic acid groups. Similarly, the trimer SA-RA-SA has 2 free carboxylic acid groups. Thus, no more than 2 molar equivalents of acetic anhydride may be used. In some embodiments, the amount of acetic anhydride is 2, 1.9, 1.8, 1.7, 1.6, 1.5, 1.4 or 1.3 molar equivalents.

In some embodiments, the acetylation step may be carried out at a temperature above 40° C. In some embodiments, the acetylation temperature is between 40° C. and the boiling point of acetic anhydride. In some embodiments, the acetylation temperature is between 40 and 90, between 40 and 100, between 40 and 110, between 80 and the boiling point of the acylation anhydride. The temperature used for the acylation-activation of the oligomers is a function of time, the longer the reaction time, the lower the temperature to be applied. It is possible to react the diacid oligomers with acetic anhydride under pressure to expedite the reaction or perform the reaction under microwave heating. These methods require tuning the reaction conditions so that the oligomers are acetylated and not deteriorated. Moreover, other acetylation methods may apply, including reaction with acetyl chloride with an acid scavenger.

The temperature may be increased following acetylation to condense the acetylated precursors to form the aforementioned dimer/trimer mixture.

The transforming into the narrow-polydispersed polymer of the invention is achieved by polymerization. Polymerization of the dimer-trimer mixture into a polymer of the invention may be achieved by heating the acetylated dimers and trimers under low pressure and elevated temperatures. In some embodiments, polymerization is achievable in vaccuo and heating. The thermal conditions may involve heating the acetylated dimer-trimer mixture to a temperature between 100 and 200, between 100 and 190, between 100 and 180, between 130 and 170, between 130 and 160, between 130 and 150, or between 130 and 140° C. In some embodiments, the temperature is between 120 and 170 or between 130 and 160° C. The reaction time is an important parameter, as the higher the reaction temperature, the shorter is the reaction time. There is a minimum time required for forming the oligomers and polymers, longer reaction time has no or little effect on the oligomer composition or polymer molecular weight. The reaction time is dependent on the batch size and the reaction conditions, including the mixing method and rate and vacuum profile applied.

In some embodiments, polymerization is achievable at high thermal conditions, as specified, under vacuum.

In some embodiments, the process comprises:
-reacting SA and RA at a temperature between 80 and 200° C. to obtain a mixture of a mono ester (SA-RA) and a diester (SA-RA-SA) of SA; and
-reacting the mixture with acetic anhydride under conditions permitting polymerization of the mono ester and diester into the polyanhydride.

In some embodiments, the process comprises:
-reacting SA and RA at a temperature between 80 and 200° C. to obtain a mixture of a mono ester (SA-RA) and a diester (SA-RA-SA) of SA; and
-reacting the mixture with acetic anhydride to acetylate the mixture of monoester and diester; and
-thermally treating the acetylated mixture under conditions permitting polymerization into the polyanhydride.

In some embodiments, the process comprises:
-reacting SA and RA in the presence of acetic anhydride at a temperature between 80 and 200° C. to obtain a mixture of a mono ester and a diester of SA, as herein; and
-thermally treating the acetylated mixture in vaccuo at a temperature between 100 and 200° C., permitting polymerization to afford the polyanhydride.

The polymer of the invention is thus a polyanhydride where the mixture or dimer and trimer dicarboxylic acids are linked to a chain by an anhydride bond. Processes of the invention exclude such processes which produce polydisperse polyanhydrides. Processes of the invention are free of steps forming or utilizing a polymer or oligomer derived from (consisting) SA or derived from (consisting) RA. One such process is a process utilizing SA and RA and disclosed in publications [1-3]. The polymer of the invention is subject of co-pending U.S. Pat. application no. 63/062,563 and any co-pending application claiming priority therefrom, each of which herein incorporated by reference.

Thus, the carrier in all its embodiments is prepared by methods or processes as herein, wherein the method or process or preparation does not comprise use of poly sebacic acid.

The invention further provides a carrier of the invention, as defined, for use in manufacturing an anticancer formulation comprising the anticancer agent. Also provided is use of the carrier or the anticancer agent for the preparation of the formulation.

The highly reproducible batch-to-batch polymer molecular weight provide improved reproducible viscosity allowing predictable injectability, highly reproducible compositions and drug release profiles, alongside a polymer degradation rate that is predictable, manageable, with a narrow standard deviation, and a high purity (minimal or no reactant impurities of acetic anhydride and anhydride molecules), the polymers of the invention are superior to those discussed in the art. Accordingly, the usability of polyanhydrides of the invention in the medical fields, e.g. as drug carriers, opens the door for a new generation of drug carriers.

Thus, in a first aspect there is provided an anticancer formulation comprising a polymer of the invention (as defined or as prepared) and at least one anticancer agent.

The anticancer formulation comprises at least one anticancer agent and a carrier in a form of a polyanhydride composed of sebacic acid (SA) and ricinoleic acid (RA), the carrier having a Mw/Mn value between 1 and 2.5. In some embodiments, the carrier is a polyanhydride of the formula —(SA—RA)n—, wherein n is an integer between 10 and 100. In some embodiments, the polyanhydride is prepared by: a. melt condensation of SA and RA to form dicarboxylic acid oligomers; b. oligomer activation with acetic anhydride; c. melt polycondensation to form a polyanhydride. The oligomer activation may be in the presence of a mole equivalent or less of acetic anhydride per carboxylic acid group, in the absence of a solvent.

As used herein, the term “formulation” refers to a pharmaceutical grade formulation or composition comprising at least one anticancer agent and a carrier that comprises or consists a polymer of the invention. Where properties of a formulation of the invention are to be modified, in some embodiments, the carrier utilized may comprise in addition to a polymer of the invention also other acceptable carriers such as, for example, vehicles, adjuvants, excipients, or diluents. The choice of using a further carrier in addition to a polymer of the invention will be determined in part by the particular anticancer agent, as well as by the particular method used to administer the composition and by the particular form of the formulation.

The anticancer formulation comprises an anticancer agent and a carrier in a form of a polyanhydride of the formula —(SA—RA)n—, wherein SA is sebacic acid and RA is ricinoleic acid, and wherein n is an integer between 10 and 100, having a Mw/Mn value (wherein Mw is the weight-average molecular weight and Mn is the number-average molecular weight) below 2.5 or below 2, or a value that is between 1 and 2.5 or 1 and 2.

In some embodiments, the polyanhydride is prepared by melt condensation of SA and RA with a mole equivalent or less of acetic anhydride per carboxylic acid group, in the absence of a solvent. In other words, the polyanhydride is not prepared by processes involving use of a solvent or polymerization of RA or SA alone.

The invention also provides use of a carrier in a form of a polyanhydride of the formula —(SA—RA)n—, wherein SA is sebacic acid and RA is ricinoleic acid, and wherein n is an integer between 10 and 100, having a Mw/Mn value (wherein Mw is the weight-average molecular weight and Mn is the number-average molecular weight) below 2.5 or below 2, or a value that is between 1 and 2.5 or 1 and 2, for preparing an anticancer formulation comprising at least one anticancer agent.

Further, an anticancer agent is provided for the preparation of an anticancer formulation comprising the anticancer agent and a carrier in a form of a polyanhydride of the formula —(SA—RA)n—, wherein SA is sebacic acid and RA is ricinoleic acid, and wherein n is an integer between 10 and 100, having a Mw/Mn value (wherein Mw is the weight-average molecular weight and Mn is the number-average molecular weight) below 2.5 or below 2, or a value that is between 1 and 2.5 or 1 and 2.

Formulations of the invention may be formed into implantable or injectable formulations. The implantable formulation may be in a form of a gel or a flowing formulation which semi-solidifies upon contact with a tissue by absorbing water to form an organo-gel. The injected polymer formulation forms a highly viscous implant that remains in the site of injection and gradually degrades and eliminates therefrom. In some embodiments, the injectable formulation is contained in a syringe and e.g., delivered using a 23 G syringe.

Formulations of the invention comprising an anticancer agent and a polymer of the invention may be formed by a variety of ways. In some cases, formulations are formed by mixing a polymer of the invention, as defined, with the at least one anticancer agent. In such cases a measurable dosage amount of the anticancer agent is mixed with an appropriate amount of the polymer to obtain a homogenous formulation. In other cases, formulations are formed by mixing the anticancer agent with the polymer precursors during preparation of the polymer.

Irrespective of the mode of preparation of formulations of the invention, the mixture of the polymer and the anticancer agent, being in a form of a paste, may be loaded into a syringe, sealed in a pouch, and sterilized by gamma irradiation. When used for intratumoral delivery, e.g., by injection, the polymer formulation increases its viscosity in the tissue as a result of interaction with the tissue aqueous environment. The anticancer agent is released in a desired controlled manner to the surrounding tissue, while the polymer implant slowly degrades and eliminates from the body shortly after the drug has been depleted.

In some embodiments, a formulation of the invention may be implanted in a subject’s body, e.g., may be introduced to a tumor site, following a surgical removal of a tumor or injected into a tumor site by a needle, either directly or through minimally-invasive laparoscopic surgery.

Generally speaking, formulations of the invention may be configured as controlled release formulations when injected to a cancer site, particularly intratumorally for weeks while being safely degraded and eliminated from the body. The term “controlled delivery” is used herein in its broadest sense to denote a formulation whereby discharge of the anticancer agent from the formulation and permeation of agent through tissues, its accessibility and bioavailability in tissues and blood circulation, and/or targeting to the specific tissues of action are modulated to achieve specific effects over time. It encompasses immediate, prolonged, and sustained delivery of the anticancer agent, drug protection against degradation, preferential metabolism, clearance or delivery to specific tissues. Controlled release of the anticancer agent included in a formulation of the invention can be obtained by several means, as known in the art.

Typically, formulations of the invention are configured as prolonged delivery or sustained delivery formulations.

The term “prolonged delivery” implies a delayed permeation and/or release of the anticancer agent from the formulation and into the tissue. In other words, in a prolonged delivery, the agent can be detected or measured in the tissue or circulation after a lag period, and in this case, after at least about 10, 20 30, 40, 50, 60, 70, 80, 90, 100, 110, 120, 130, 140, 150, 160, 170, 180 min and further after at least about 3 h, 4 h, 5 h, 6 h, 7 h, 8 h, 9 h, 10 h or more after administering. The prolonged delivery also applies to target organs and tissues with additional lag of at least about 10, 20 30, 40, 50, 60, 70, 80, 90, 100, 110, 120, 130, 140, 150, 160, 170, 180 min and further after at least about 3 h, 4 h, 5 h, 6 h, 7 h, 8 h, 9 h, 10 h or more after administering.

The term “sustained delivery” implies a profile of continued released and/or permeation of the agent from the formulation and into the tissue or circulation, or in other words, that the relates and/or permeation of the agent from the formulation and into the tissue or circulation reaches a plateau or a steady state after at least about 10, 20 30, 40, 50, 60, 70, 80, 90, 100, 110, 120, 130, 140, 150, 160, 170, 180 min and further after at least about 3 h, 4 h, 5 h, 6 h, 7 h, 8 h, 9 h, 10 h or more after administering, and that the plateau or the steady state persists for at least about 1 h, 2 h, 3 h, 4 h, 5 h, 6 h, 7 h, 8 h, 9 h, 10 h, 11 h, 12 h, 13 h, 14 h, 15 h, 16 h, 17 h, 17 h, 18 h, 19 h, 20 h or more after.

The anticancer agent may be any cytotoxic agent, protein or nucleotide-based biological drug used in treating cancer. As known in the art, “cancer” refers to any malignant condition, namely to a severe and progressively worsening disease which potentially poses a mortal threat to the suffering subject. The malignancy, as in malignant neoplasm, and malignant tumor, are used synonymously with cancer, and also prefix other oncology terms such as malignant ascites, malignant transformation.

When used to fight or manage a malignant proliferative disease or disorder, e.g., cancer, the anticancer formulations presented herein can be used to treat a wide spectrum of cancers (neoplasms), such as blastoma, carcinoma, lymphoma, leukemia, sarcoma, mesothelioma, glioma, germinoma, choriocarcinoma, melanoma, glioblastoma, colon, head and neck, GI and lymphoid malignancies as well as any other neoplastic disease or disorder, collectively referred to as cancer. Other examples of cancer which can be treated using compounds according to some embodiments of the present invention include, but are not limited to, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In some embodiments, formulations of the invention are used for managing a solid cancer. Solid cancers appear in many forms, for example, brain cancer, breast cancer, prostate cancer, head and neck sarcoma, and skin cancer. One form of skin cancer is melanoma. Melanoma is the most aggressive form of skin cancer and is notoriously resistant to current modalities of cancer therapy.

The anticancer agent used in accordance with the invention may be a general anticancer agent or one specifically designed to treat or prevent a particular type of cancer. The anticancer agent may by selected amongst cytotoxic agents, chemotherapeutic agents such as alkylating agents, intercalating drugs, topoisomerase inhibitors, antimetabolites, and antimitotic drugs, as well as kinase inhibitors, monoclonal antibiotics and others.

Non-limiting examples of anticancer agents include alkylating agents such as altretamine, bendamustine, busulfan, carmustine, chlorambucil, cyclophosphamide, dacarbazine, ifosfamide, lomustine, mechlorethamine, melphalan, procarbazine, streptozocin, temozolomide, thiotepa and trabectedin; platinum complexes such as carboplatin, cisplatin and oxaliplatin; antibiotics and cytotoxic agents such as bleomycin, dactinomycin, daunorubicin, doxorubicin, epirubicin, idarubicin, mitomycin, mitoxantrone, plicamycin and valrubicin; antimetabolites; antifolates such as methotrexate, pemetrexed, pralatrexate and trimetrexate; purine analogues such as azathioprine, cladribine, fludarabine, mercaptopurine and thioguanine; pyrimidine analogues such as azacitidine, capecitabine, cytarabine, decitabine, floxuridine, fluorouracil, gemcitabine and trifluridine or tipracil; biologic response modifiers such as aldesleukin (Il-2), denileukin diftitox and interferon gamma; histone deacetylase inhibitors such as belinostat, panobinostat, romidepsin and vorinostat; Antiandrogens such as abiraterone, apalutamide, bicalutamide, cyproterone, enzalutamide, flutamide and nilutamide; antiestrogens such as anastrozole, exemestane, fulvestrant, letrozole, raloxifene, tamoxifen and toremifene; gonadotropin releasing hormone analogues such as degarelix, goserelin, histrelin, leuprolide and triptorelin; peptide hormones such as lanreotide, octreotide and pasireotide; monoclonal antibodies such as alemtuzumab, atezolizumab, avelumab, bevacizumab, blinatumomab, brentuximab, cemiplimab, cetuximab, daratumumab, dinutuximab, durvalumab, elotuzumab, gemtuzumab, inotuzumab ozogamicin, ipilimumab, mogamulizumab, moxetumomab pasudotox, necitumumab, nivolumab, ofatumumab, olaratumab, panitumumab, pembrolizumab, pertuzumab, ramucirumab, rituximab, tositumomab and trastuzumab; protein kinase inhibitors such as abemaciclib, acalabrutinib, afatinib, alectinib, axitinib, binimetinib, bortezomib, bosutinib, brigatinib, cabozantinib, carfilzomib, ceritinib, cobimetinib, copanlisib, crizotinib, dabrafenib, dacomitinib, dasatinib, duvelisib, enasidenib, encorafenib, erlotinib, gefitinib, gilteritinib, glasdegib, ibrutinib, idelalisib, imatinib, ivosidenib, ixazomib, lapatinib, larotrectinib, lenvatinib, lorlatinib, midostaurin, neratinib, nilotinib, niraparib, olaparib, osimertinib, palbociclib, pazopanib, pexidartinib, ponatinib, regorafenib, ribocicib, rucaparib, ruxolitinib, selumetinib, sonidegib, sorafenib, sunitinib, talazoparib, trametinib, vandetanib, vemurafenib, vismodegib and zanubrutinib; taxanes such as cabazitaxel, docetaxel and paclitaxel; topoisomerase inhibitors such as etoposide, irinotecan, teniposide and topotecan; vinca alkaloids such as vinblastine, vincristine and vinorelbine; and other neoplastic drugs such as asparaginase (pegaspargase), bexarotene, eribulin, everolimus, hydroxyurea, ixabepilone, lenalidomide, mitotane, omacetaxine, pomalidomide, tagraxofusp, telotristat, temsirolimus, thalidomide and venetoclax. Each of the aforementioned agents constitutes a separate embodiment of the invention.

In some embodiments, the anticancer agent is paclitaxel, cisplatin or tamoxifen.

Further provided by the invention are methods of treatment or prevention utilizing formulations of the invention.

In one aspect, there is provided a method for treating or delaying or preventing the progression of a proliferative disorder, e.g., cancer, the method comprising administering an effective amount of an anticancer agent in a formulation of the invention, as described herein, to a subject in need thereof.

The term “treatment” as used herein refers to the administering of a therapeutic amount of the formulation of the present invention which is effective to ameliorate undesired symptoms associated with a disease, to prevent manifestation of such symptoms before they occur, to slow down progression of the disease (also referred to herein as “delaying the progression”), slow down deterioration of symptoms, to enhance onset of remission period, slow down irreversible damage caused in a progressive chronic stage of the disease, to delay onset of said progressive stage, to lessen severity or cure the disease, to improve survival rate or more rapid recovery, or to prevent the disease from occurring or a combination of two or more of the above.

The term “effective amount” as used herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

The anticancer agent may be present in formulations of the invention in an amount or dose, which will depend on a variety of considerations known to those versed in the field. Without wishing to be bound by any dose amounts, typically the anticancer agent may be present in an amount between 0.1 and 75% w/w, depending on the potency of the drug, the volume of formulation configured for, e.g., injection, and the desired release profile. The hydrophobic nature of the polymer of the invention may protect, in part, the incorporated drug from being deteriorated due to light interaction, oxidation or hydrolysis during storage and in patient. The pasty polymer can be injected into tumor or tissue or spread on disease surface such as the lungs, colon and other tissues with spread cancerous cells and tissues. The distribution of the active agent into the surrounding cancerous tissue, after, e.g., intratumoral injection, is dependent on the tissue properties; usually the diffusion of the agent can reach 15 mm, or more, from the injection site. The spread of active agent can be improved by adding agents that enhance tissue penetration such as Azone, isopropyl myristate, decyl oleate, oleyl alcohol and triacetin. The polymer containing the drug can be dispersed in water for injection to form a dispersion that can be injected or spread into and onto tissue. The release of active agents can be of zero order or first order profile for periods from a few days to about 8 weeks.

Formulations of the invention may be delivered by a variety of ways. In some embodiments, the effective amount of the anticancer agent is administrated by one or more of the following routes transmucosal, transnasal, intestinal, parenteral, intramuscular, subcutaneous, intramedullary injections, intrathecal, direct intraventricular, intravenous, intraperitoneal, intranasal, or intraocular injections.

In some embodiments, the formulation is administered by injection.

In some embodiments, the formulation is administered by injection into a tumor (intertumorally).

In some embodiments, the formulation is administered by implanting same into a tissue or an organ.

The invention thus provides a method of administering a formulation according to the invention, wherein the method comprises administering said formulation to a subject by:
(i) Injection into a tumor site;
   a. prior to removal of the tumor;
   b. following or at a time period after removal of the tumor;
   c. following a recurrence of the tumor at the treated site;
(ii) Implanting a device comprising or consisting a formulation of the invention at the vicinity or in proximity to a tumor site;
   a. Prior to removal of the tumor;
   b. Following or at a time period after removal of the tumor;
   c. Following recurrence of the tumor at the treated site;
(iii) Layering a formulation onto a tumor or a tissue at a tumor site following removal of a tumor;
(iv) Delivering a formulation of the invention via Laparoscopy;
(v) Any of the above in combination with chemotherapy or radiation;
(vi) Coating or loading of the formulation onto or into a device or implant to be in contact or at the vicinity or the tumor site;
(vii) Injection to the body for achieving systemic administration of anticancer agents for systemic extended release of the anticancer drugs.

As used herein, the terms “vicinity” and “proximity” relate to a distance from a tumor or diseased tissue to be treated. The distance ranges from the site of tumor or tissue to a distance that is 1 to 10 centimeters therefrom. Thus the terms relate to a distance of between zero and 10 cm, wherein zero cm designates a center of the tumor or diseased tissue. In some embodiments, administration is directly to or into the tumor. In other embodiments, administration is to the surrounding of the tumor at a distance that is up to 10 cm therefrom.

Also provided is a kit comprising the carrier, as defined or as prepared and an anticancer agent. In some embodiments, the carrier and agent are separately contained, namely each contained in a different vessel. In some embodiments, they are contained together. In some embodiments, the kit is a syringe or comprises a syringe. The kit will also contain instructions of use.

Methods of use and uses according to the invention utilize a carrier in a form of a polyanhydride of the formula —(SA—RA)n—, wherein SA is sebacic acid and RA is ricinoleic acid, and wherein n is an integer between 10 and 100, having a Mw/Mn value (wherein Mw is the weight-average molecular weight and Mn is the number-average molecular weight) below 2.5 or below 2, or a value that is between 1 and 2.5 or 1 and 2.

In some embodiments, the carrier is prepared by any of the processes disclosed herein.

In some embodiments, formulations used in accordance with the invention comprise the anticancer agent, as defined, and a carrier, as defined, wherein the carrier is prepared by a process comprising melt polycondensation of RA and SA in presence of an amount of acetic anhydride not exceeding a mole equivalent thereof per each free carboxylic acid group and in absence of a solvent.

### BRIEF DESCRIPTION OF THE DRAWINGS

The invention may be more clearly understood upon reading of the following detailed description of non-limiting exemplary embodiments thereof, with reference to the following drawings, in which:

FIG. 1 is a synthetic scheme of a polyanhydride carrier of the present invention.

FIG. 2 is graphical representation illustrating in vitro cumulative release of cisplatin from PSA:RA 3:7 loaded with cisplatin at 37℃. During the 25 days, ~75 % and ~10 % of the incorporated drug was released from 0.5 and 20% and 5 and 10% (w/w) of the drug formulation.

FIGS. 3A-3D show FIG. 3A) overall release of drug from 50 mg sample in 50 mL buffer,

FIG. 3B) overall release of drug from 100 mg sample in 50 mL buffer, FIG. 3C) overall release of drug from 200 mg sample in 50 mL buffer, FIG. 3D) comparison of drug release amount mg) per day.

FIG. 4 shows change in body weight of subjects in the study.

FIG. 5 shows comparison of IV chemotherapy to cisplatin-polymer over kidneys performance and bodyweight (FIG. 4).

FIG. 6 shows a comparison of IV chemotherapy to TumoCure over blood count.

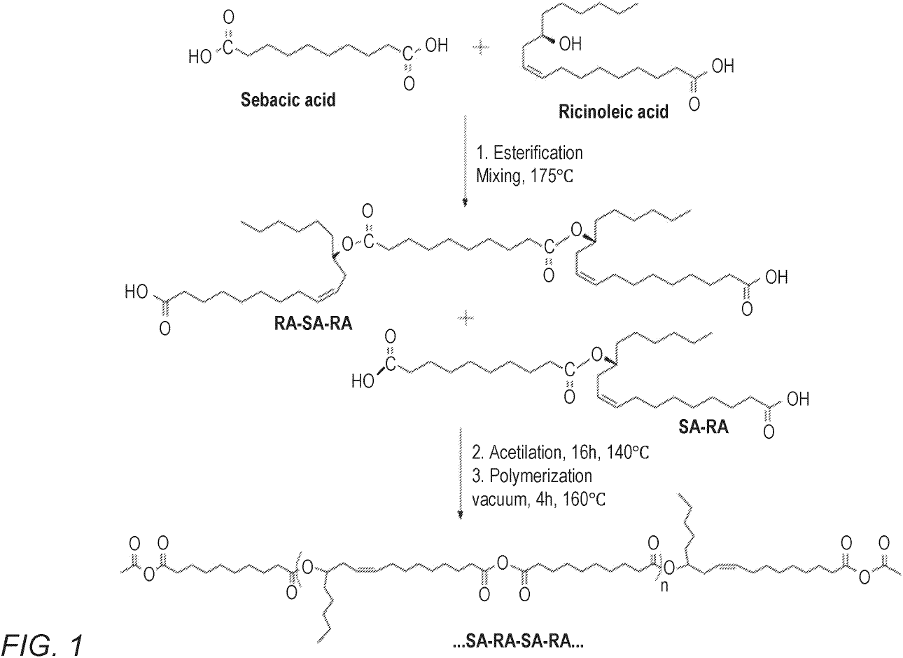

### DETAILED DESCRIPTION OF THE INVENTION

### Example 1: Controlled Synthesis of Oligomers of Different Type of Dicarboxylic Acid and Hydroxy Acids Forming a Carrier According to the Invention

Aim: development of an alternative method to synthesis of oligomers of different type of dicarboxylic acid and hydroxy acids.

Materials: Suberic acid (SUA) and dodecanedioic acid (DDDA) were used as received. Ricinoleic acid (RA) was prepared from the hydrolysis of castor oil as described in the synthesis part.

### Spectral Analysis

¹H and ¹³C NMR spectra were obtained on a Varian 300 MHz NMR spectrometer using CDCl3 as solvent containing tetramethylsilane as shift reference. Fourier transform infrared (FTIR) spectroscopy was performed using a Smart iTR ATR sampling accessory for Nicolet iS10 spectrometer with a diamond crystal (Thermo Scientific, Massachusetts).

Preparation of ricinoleic acid from castor oil: In a 1000 mL round bottom flask, 48 g of KOH was dissolved in 400 mL of ethanol by heating (65° C.). Then, 200 g of castor oil was added to it and mixed them properly. The mixture was then refluxed for 2 hr at 140° C. with continuous staring. After the reflux, the solvent was evaporated by evaporator. Then 200 mL of double distilled water, 150 mL diisopropyl ether, and 150 mL H3PO4 were added and the total mixture was transferred to a separating funnel. It was then repeatedly washed with double distilled water (3-5 times, 200 mL each time) until the pH of the aqueous phase ~4. Then the organic phase was collected through sodium phosphate and evaporated to dryness to obtain pure 185 g of Ricinoleic acid (yield 92.5%), confirmed by ¹H NMR.

Synthesis of SUA-RA and DDDA-RA oligomers: SUA-RA and DDDA-RA oligomers were synthesized by esterification reaction of suberic acid and dodecanedioic acid with ricinoleic acid at 170° C. In a round bottom flask, 15 g of SUA, 15 g of RA and catalytic amount (1%) of phosphoric acid were taken and heated to 170° C. for 5 hours under nitrogen. Then another 15 g of RA was added to the round bottom flask and continued to heat for another 4 hours under nitrogen swift. Finally another 5 g of RA was added and again continued to heat over night with mixing under vacuum to yield SUA-RA oligomer with 30:70 ratios of SUA and RA which was characterized by ¹H NMR. DDDA-RA oligomer with 30:70 ratios of DDDA and RA was synthesized following the same procedure and was also characterized by ¹H NMR.

Discussion of the results: Two different oligomers are synthesized using two different dicarboxylic acid and hydroxy acids. RA is esterified with SUA or DDDA under melt and vacuum condition where H3PO4 is used as catalyst. Under this reaction condition 100% of the RA is consumed in the esterification reaction with SUA or DDDA which is confirmed from the ¹H NMR as the signal at 3.6 ppm for the alcoholic proton is gone astray after the final step of esterification. Furthermore, self-condensation of RA in this protocol (via step by step addition of RA to SUA or DDDA) is also avoided; evidence form ¹H NMR, as there is no signal at 4.1 ppm. Hence this process gives a well-defined SUA-RA or DDDA-RA oligomers without any residual or self-condensed RA.

### Example 2: Synthesis of Poly(ester-anhydride) Approaching From an Alternative Method

The objective is the development an alternative method to synthesis of biodegradable copolymer of poly(ester-anhydride). Here the focus is on two features:
1) Use of sebacic acid (SA) and ricinoleic acid (RA) or 12-hydroxystearic acid (HSA) to prepare SA-RA or SA-HSA oligomers by direct condensation.
2) Use of fewer amounts (1:1 equivalent or less) of acetic anhydride to activate the oligomers for polymerization.
3) Control the molecular weight of poly(ester-anhydride) depending upon amount the acetic anhydride used for the pre-polymerization step.

Materials: Sebacic acid (SA, 99% pure; Aldrich, USA), 12-hydroxystearic acid (HSA) and acetic anhydride (Merck, Germany) were used as received. Ricinoleic acid (RA) was prepared from the hydrolysis of castor oil as described in the synthesis part.

Spectral analysis^{:} ¹H and ¹³C NMR spectra were obtained on a Varian 300 MHz NMR spectrometer using CDCl3 as solvent containing tetramethylsilane as shift reference. Fourier transform infrared (FTIR) spectroscopy was performed using a Smart iTR ATR sampling accessory for Nicolet iS10 spectrometer with a diamond crystal (Thermo Scientific, Massachusetts).

Molecular weight determination: The molecular weights were determined by gel permeation chromatography (GPC) system, Waters 1515. Isocratic HPLC pump with a Waters 2410 refractive index detector, a Waters 717 plus autosampler, and a Rheodyne (Cotati, CA) injection valve with a 20 µL-loop. The samples were eluted with CHCl3 (HPLC grade) through linear Styragel HR5 column (Waters) at a flowrate of 1 mL/min. The molecular weights were determined relative to polystyrene standards.

Synthesis and Characterization: SA-RA oligomers: SA-RA oligomers were synthesized by heating ricinoleic acid and sebacic acid at 175° C. In a round bottom flask, 30 g of SA, 30 g of RA and catalytic amount (0.1%) of phosphoric acid were taken and heated to 170° C. for 5 hours under nitrogen. Then another 30 g of RA was added to the round bottom flask and continued to heat for another 4 hours under nitrogen swift. Finally, another 10 g of RA was added and again continued to heat over night with mixing under vacuum to yield SA-RA oligomer with 30:70 ratios of SA and RA which was characterized by ¹H NMR and FTIR. The SA-RA oligomers of different ratios were also prepared by the same process and characterized by ¹H NMR. The details are given in the Table 1 below.

TABLE 1

SA-RA oligomers

| SA-RA ratio | SA | RA | | |
|---|---|---|---|---|
| | | 1^{st} Step, 170° C., 5 hrs, N2 | 2^{nd} Step 170° C., 4 hrs, N2 | 3^{rd} Step 170° C., Overnight, Vacuum |
| 20:80 | 10 g | 17.5 g | 17.5 g | 5 g |
| 25:75 | 12.5 g | 16.25 g | 16.25 g | 5 g |
| 35:65 | 17.5 g | 13.75 g | 13.75 g | 5 g |

### SA-HAS Oligomers

SA-HSA oligomers were also synthesized by heating 12-hydroxystearic acid and sebacic acid at 175° C. In a round bottom flask, 15 g of SA, 15 g of HSA and catalytic amount (0.1%) of phosphoric acid were taken and heated to 170° C. for 5 hours under nitrogen. Then another 15 g of HSA was added to the round bottom flask and continued to heat for another 4 hours under nitrogen swift. Finally, another 5 g of HSA was added and again continued to heat over night with mixing under vacuum to yield SA-HSA oligomer with 30:70 ratios of SA and HSA which was characterized by ¹H NMR and FTIR. The SA-HSA oligomers of 20:80 ratios were also prepared by the same process. The details are given in the Table 2 below.

TABLE 2

SA-RA oligomers

| SA-has ratio | SA | HSA | | |
|---|---|---|---|---|
| | | 1^{st} Step 170° C., 5 hrs, N2 | 2^{nd} Step 170° C., 4 hrs, N2 | 3^{rd} Step 170° C., Overnight, Vacuum |
| 20:80 | 10 g | 17.5 g | 17.5 g | 5 g |

### Poly(SA-RA)

In a typical synthesis, 10 g of 20:80, 25:75, 30:70, 35:65 ratio of SA-RA oligomers were melt individually at 140° C. under nitrogen atmosphere. Then 1:5 equivalent of acetic anhydride was added to the molten SA-RA oligomers and refluxed at 140° C. for 60 min. Excess acetic anhydride or acetic acid was evaporated. The residue was then subjected to melt condensation at 160oC under 10mbar for 4 hours. The SA-RA oligomer of 30:70 ratios was also polymerized under same procedure where different amount (1, 0.7, 0.5, 0.35, 0.25, 0.15 equivalent) of acetic anhydride was used (refluxed at 140° C., overnight) to use fewer amount of acetic anhydride and make a control over the molecular weight

### Poly(SA-HSA)

Following the same procedure as poly(SA-RA), 10 g of 20:80 and 30:70 ratio of SA-HSA oligomers were melt individually at 140° C. under nitrogen atmosphere. Then 1:5 equivalent of acetic anhydride was added to both of the molten SA-HSA oligomers and refluxed at 140° C. for 60 min. Excess acetic anhydride or acetic acid was evaporated. The residue was then subjected to melt condensation at 160° C. under vacuum (~10 m bar) for 4h.

### Discussion of the Results

Two kinds of poly(ester-anhydride) copolymers were synthesized through solvent free melt polycondensation process where directly sebacic acid is used to synthesis the SA-RA or SA- HSA oligomers instead of using poly(SA) as starting material. RA or HAS is esterified with SA under melt and vacuum condition where in some cases, up to 1% H₃PO₄ is used as catalyst. Under this reaction condition 100% of the RA or HSA is consumed in the esterification reaction with SA which is confirmed from the ¹H NMR as the signal at 3.6 ppm for the alcoholic proton is gone astray after the final step of esterification. Furthermore, self-condensation of RA or HSA in this protocol (via step by step addition of RA or HAS to SA) is also avoided; evidence form ¹H NMR, as there is no signal at 4.1 ppm. Hence this process gives a well-defined SA-RA or SA- HSA oligomers without any residual or self-condensed RA or HSA. The proton of the esterified polymer chemical shift observed at ~4.8 ppm. Two protons adjacent to the ester bonds and anhydride bonds arise at 2.43 ppm and 2.33 ppm, respectively.

The molecular weight of the as-synthesized polymers is measured by GPC. The details of the molecular weight and disparity are given in the below Table 3 and control over molecular weight depending upon the acetic anhydride used.

TABLE 3

molecular weight and disparity of polymers of the invention

| S1. No | polymer | Molecular weight (M_{w}) Daltons | polydispersity (PD) |
|---|---|---|---|
| 1 | Poly(SA-RA) with 20:80 ratio, using 1:5 w/w acetic anhydride | 17091 | 3.01 |
| 2 | Poly(SA-RA) with 25:75 ratio, using 1:5 w/w acetic anhydride | 18793 | 3.07 |
| 3 | Poly(SA-RA) with 30:70 ratio, using 1:5 w/w acetic anhydride | 12335 | 2.85 |
| 4 | Poly(SA-RA) with 35:65 ratio, using 1:5 w/w acetic anhydride | 18558 | 3.02 |
| 7 | Poly(SA-RA) with 30:70 ratio, using 0.5 equivalent acetic anhydride | 4841 | 1.72 |
| 8 | Poly(SA-RA) with 30:70 ratio, using 0.35 equivalent acetic anhydride | 3296 | 1.51 |
| 9 | Poly(SA-RA) with 30:70 ratio, using 0.25 equivalent acetic anhydride | 2357 | 1.35 |
| 10 | Poly(SA-RA) with 30:70 ratio, using 0.15 equivalent acetic anhydride | 1856 | 1.24 |
| 11 | Poly(SA-HSA) with 20:80 ratio, using 1:5 w/w acetic anhydride | 15498 | 3.18 |
| 12 | Poly(SA-HSA) with 30:70 ratio, using 1:5 w/w acetic anhydride | 17630 | 3.33 |

### Example 3: Synthesis of poly(SA-RA) With Reduced Reaction Time

Aim: The aim of the project is to monitor the synthesis process via ¹H NMR of biodegradable copolymer of poly(sebacic acid - ricinoleic acid) to reduce the reaction time. Materials: Sebacic acid (SA, 99% pure; Aldrich, USA) was used as received. Ricinoleic acid (RA) was prepared from the hydrolysis of castor oil as described in the synthesis part.

Spectral analysis: ¹H NMR spectra were obtained on a Varian 300 MHz NMR spectrometer using CDCl3 as solvent. Fourier transform infrared (FTIR) spectroscopy was performed using a Smart iTR ATR sampling accessory for Nicolet iS10 spectrometer with a diamond crystal (Thermo Scientific, Massachusetts).

Molecular weight determination: The molecular weights were determined by gel permeation chromatography (GPC) system, Waters 1515. Isocratic HPLC pump with a Waters 2410 refractive index detector, a Waters 717 plus autosampler, and a Rheodyne (Cotati, CA) injection valve with a 20 µL-loop. The samples were eluted with CHCl₃ (HPLC grade) through linear Styragel HR5 column (Waters) at a flowrate of 1 mL/min. The molecular weights were determined relative to polystyrene standards.

Synthesis of SA-RA oligomer: SA-RA oligomers were synthesized by heating ricinoleic acid and sebacic acid at 170° C. In a round bottom flask, 15 g of SA, 15 g of RA and catalytic amount (0.1%) of phosphoric acid were taken and heated to 170° C. for 2 hours under nitrogen. Then another 15 g of RA was added to the round bottom flask and continued to heat for another 2 hours under vacuum for 15 min followed by nitrogen swift. Finally, 5 g of RA was added and again continued to heat for another 8 hours under vacuum to yield SA-RA oligomer with 30:70 w/w ratio of SA and RA which was characterized by ¹H NMR.

poly(SA-RA): In a typical synthesis, 10 g of SA-RA oligomer with 30:70 ratios were melted at 140° C. under nitrogen atmosphere. Then 1 equivalent of acetic anhydride with respect to the acid in the oligomer was added to the molten SA-RA oligomer and refluxed at 140° C. for 2 hours. Excess acetic anhydride or acetic acid was evaporated. The residue was then subjected to melt condensation at 160° C. under vacuum (~10 m bar) for 4 hours.

### Discussion of the Results

RA is esterified with SA under melt and vacuum condition with no other additives. The addition of an acid, H3PO4 as catalyst was not needed as full conversion of the ester dimers and trimer was achieved without any addition of an acid. Under this reaction condition 100% of the RA is consumed within 12 hours in the esterification reaction with SA. This is confirmed by ¹H NMR, thus, as the signal at 3.6 ppm for the alcoholic proton is gone astray after the final step of esterification. Furthermore, self- condensation of RA in this protocol (via step by step addition of RA to SA) is also avoided; evidence form ¹H NMR, as there is no signal at 4.1 ppm. Then the oligomer was polymerized by refluxing at 140° C. with 1 equivalent of acetic anhydride for 2 hours followed by heating at 160° C. under vacuum for 4 hours. The molecular weight of the polymer is measured by GPC and compared with the polymer that is synthesized from the same SA-RA oligomer with 30:70 ratios by refluxing at 140° C. with 1 equivalent of acetic anhydride for overnight followed by heating at 160° C. under vacuum for 4 hours. It is noticed that both the process gives almost same molecular weight of the polymers (~11500 Daltons).

### Example 7: Cisplatin Loaded P(SA-RA) Injectable Formulation

Material and Methods: Materials: a) Cisplatin (99.99%) Lot# A0402164, CAS#15663-27-1, was obtained from ACROS ORGANICS; b) PSA:RA 3:7 (Mw 11675, PDI 2.63) was synthesized in the lab; c) orthophenylene diamine (OPDA); d) sodium chloride; and Dimethylformamide (DMF) was obtained from Sigma Aldrich.

### Procedure

Cisplatin stock solution of 1 mg/mL was prepared in phosphate buffer pH 7.2 containing 1% NaCl. From the stock solution different dilutions of cisplatin was prepared ranging in between 0.5 to 5 µg/mL. Then 1 mL of 1.2 mg/mL of OPDA solution in DMF was added and heated at 90° C. for 20 min to obtain a light green color solution. The prepared colored solutions were cooled to room temperature and measured at 705 nm using a UV Visible spectrophotometer.

### Results

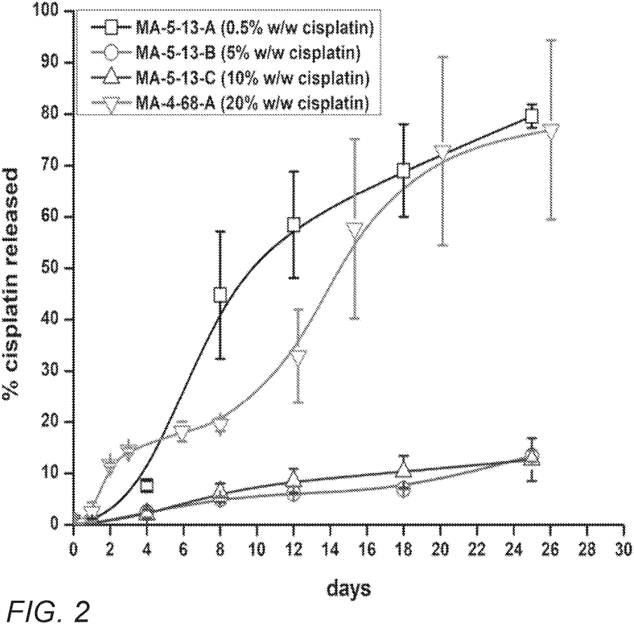

The presence of cisplatin in the PSA:RA did not affect its injectability properties. Further, the formulations became hard gels instantly after addition of the buffer medium. The release profile from the four formulations is given in FIG. 2. FIG. 2 presents in vitro cumulative release of cisplatin from PSA:RA 3:7 loaded with cisplatin at 37° C. During the 25 days, ~75 % and ~10 % of the incorporated drug was released from 0.5 and 20% and 5 and 10% (w/w) of the drug formulation.

The presence of high amount of cisplatin in PSA:RA does not influence the gelling properties of PSA:RA. There is no burst release observed in the formulation after addition of 50 mL release medium under 175 rpm shaking and at 37° C.

### Example 8: In Vivo Testing of the Cisplatin Formulation for Treating Head and Neck Cancer, Radiotherapy Effect

In this study, 3 experimental groups were used, 6 nude mice per group. All mice were injected with 600000 tumor cells. After 20 days from cell injection, the mice received either an IP injection of cisplatin solution or subcutaneous injection of polymer only as reference and polymer-cisplatin formulation (10 microliter of 0.5% cisplatin). Cisplatin solutions were injected IP for 4 consecutive weeks, a total of 4 administrations. The animals were irradiated with 8 Gy at day 21 and day 24. The experiment was terminated at day 50. The animal groups treated with drug free polymer and cisplatin solution did not show any effect on the tumor and a significant increase in the tumor size was obtained. However, the treated group with polymer-cisplatin and irradiation diminished completely the tumor.

Polymer-cisplatin was effective in reducing the rate of tumor growth over time. The rate of tumor growth reduces as a function of cisplatin-polymer dose.

Paclitaxel delivery: Paclitaxel powder (100 mg) was mixed in poly(SA-RA)20:80 (900 mg) to form a uniform white paste which was loaded in a one ml syringe. The formulation was added in 50 ml conical plastic tubes, 100 mg in each, and phosphate buffer pH 7.4 containing 0.1% w/w SDS, was added (50 ml) the vial was left at 37° C. with shaking and the solution was replaced periodically after 1, 3, 7, 14, 21 and 28 days. Paclitaxel released to the media was determined by HPLC. A constant release of about 50% of the loaded drug was released over 28 days. The remaining paclitaxel in the polymer residue accounted to most of the expected content.

Tamoxifen delivery: Solid poly(SA-RA)70:30 w/w implants loaded with 10 and 20% drug were prepared by melt process where the drug was mixed in the molten polymer and after through mixing to form a uniform melt, the formulation was casted into thin road using a mold. All experiments with tamoxifen citrate were carried out in the dark, as the drug is highly photosensitive. The drug was loaded in concentrations of 10 and 20% w/w into the polymer. Cylindrical implants were prepared by the incorporation of uniformly mixed tamoxifen citrate and poly(SA-RA) 70 : 30 w/w into a cylindrical mold of 1.5 mm in diameter. Drug content in the implants were determined by HPLC method. Tamoxifen was constantly released for more than 4 weeks. A pasty injectable polymer formulation was prepared and characterized using poly(SA-RA) 30:70 w/w pasty polymer, instead of the solid poly(SA-RA) 70:30 carrier.

Other anticancer agents: methotrexate, doxorubicin, temozolomide, acriflavine and nintedanib, alone or in combination with other drugs were incorporated in the pasty polymer by simple mixing of the drug powders in the polymer at room temperature and loading in syringes. The pasty formulation was tested for in vitro release where the drugs were released constantly over 30 days.

### Example 9 Toxicity of Polymer Cisplatin

Poly(SA:RA) 30:70 prepared as described above with a molecular weight of Mw=9400 and Mn=8200 (determined by GPC using polystyrene standards) was used in this study. The polymer was an injectable viscous gel. The viscosity was not affected at 20% drug loading. The polymer and cisplatin loaded formulations were subjected to sterilization by g-irradiation at a dose suitable for medical applied devices and combination devices, 35 to 45 kGy. No change in polymer molecular weight, drug content and drug release was observed. This indicates that the polymer and cisplatin formulations are stable to gamma irradiation sterilization.

Formulation: Two formulations were prepared, containing 10% and 20% (w/w) drug. Once prepared, they were transferred into glass pre-filled syringes, then shipped to sterilization under gamma irradiation.

In-vitro release: In vitro release tests were performed in triplicated, where 50, 100 and 200 mg of both formulations, 10 & 20% (w/w), transferred into 50 mL glass vials. To each vial 50 mL of PBS buffer (100 mM, pH=7/4 and 1% NaCl) were added, then replaced daily. The discarded solution was tested for drug content using spectrophotometric methods to assess amount (mg) of drug released.

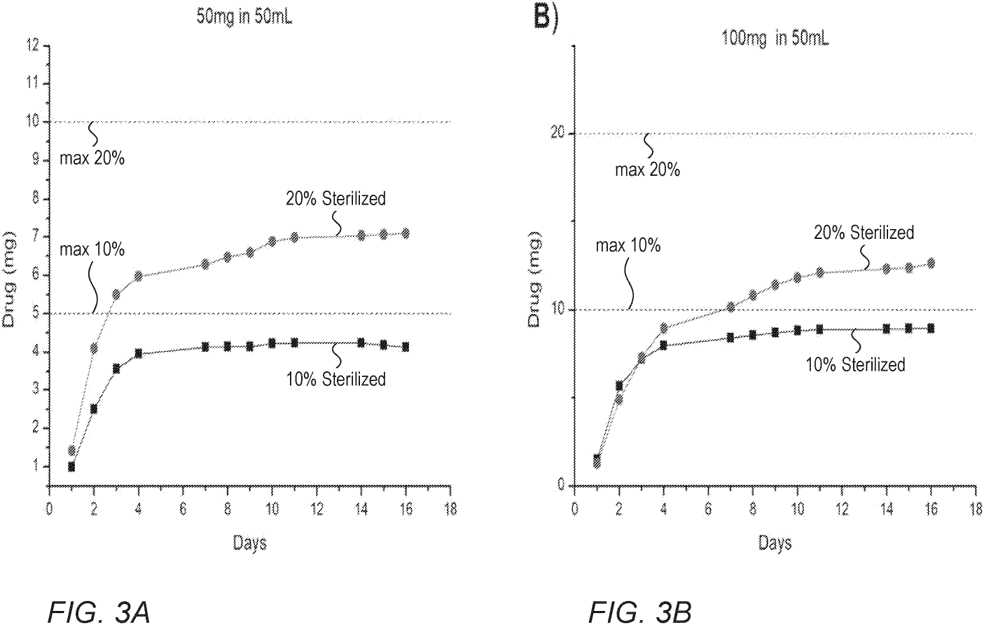

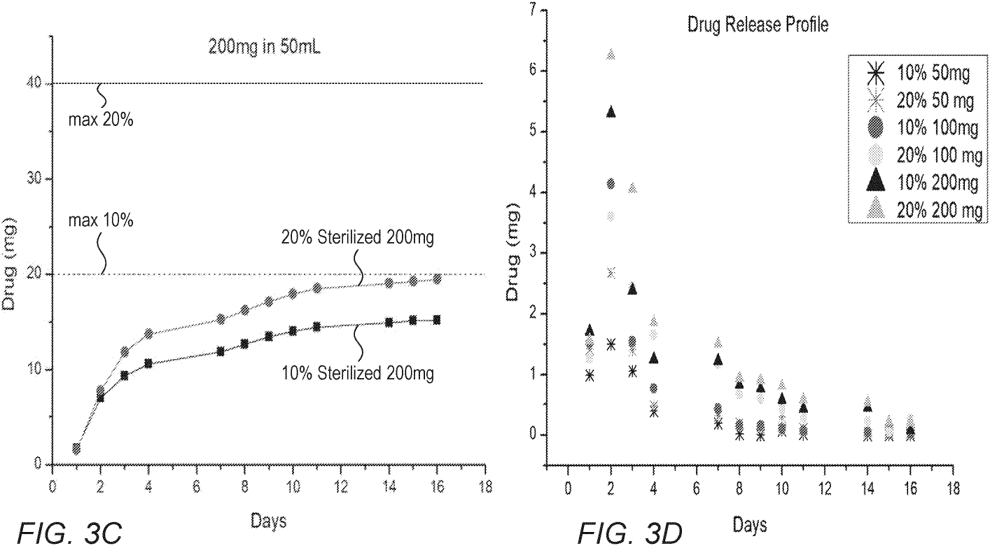

As can be seen from FIGS. 3A-C, the higher the surface area (lower amount introduced) the faster it releases the drug, which means that the polymer is more prone to hydrolysis i.e. degrades faster. Moreover, the maximal release, is regardless of the amount tested, and it occurs during the second and third days from injection as can be seen in FIG. 3D.

Pharmacokinetics trials on Rats: Due to dose limitation, we focused on 10% formula, where we injected and compared the sterilized 10% formula to the IV drug and PSARA alone, in order to assess the MTD, PK and the effect over organs and body in general.

Polymer-cisplatin was injected subcutaneously to mice at the amount 10, 20, 40 µL per mice that is 4, 8, 16 mg cisplatin/Kg. As controls, 50µL of the drug free polymer was injected and 200 µL IV injection at a dose of 5 mg/kg. The mice were observed daily for morbidity and mortality, and daily for general clinical signs (cage side observation): changes in the skin, fur, eyes, nose, mouth, head, respiration, urine, feces, locomotor, and overall wellness.

An animal showing one of the humane endpoints according to Clinical Signs″, SOP-06-019, were euthanized and subjected to the termination procedure. Blood collection for cisplatin content was at 0(baseline), 1 h, 4 h, 8 h, 24 h,48 h,96 h, 168 h, thereafter once weekly until week 4. Blood for CBC and Biochemistry was at: 0(Baseline), 24 h, 72 h, 168 h thereafter once weekly until sacrifice. For the IV injected mice, plasma was collected at 0(baseline), 5 m, 15 m, 30 m, 1 h, 2 h, 4 h, 8 h, 24 h, 48 h, 72 h, and 96 h. Whole blood and Blood Biochemistry was at 0(Baseline), 24h and 96h. Whole blood was collected from each animal and treated according to common procedures. Urine collection amount was recorded at 0-6, 12-24, 48-72h, 72-96h, 96-120h and once weekly thereafter.

### Results

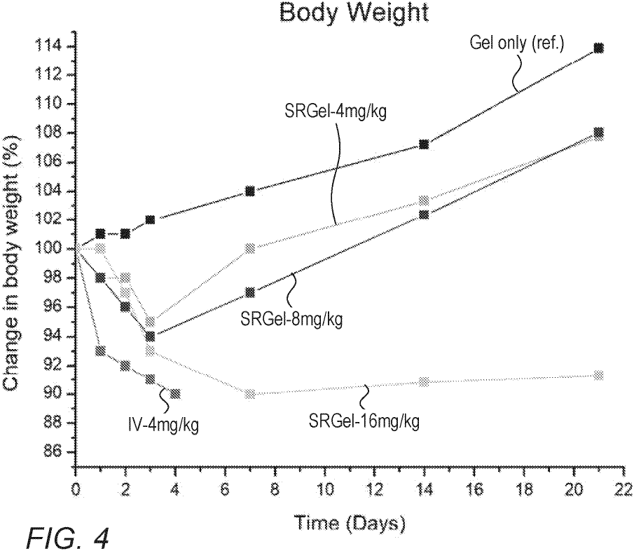

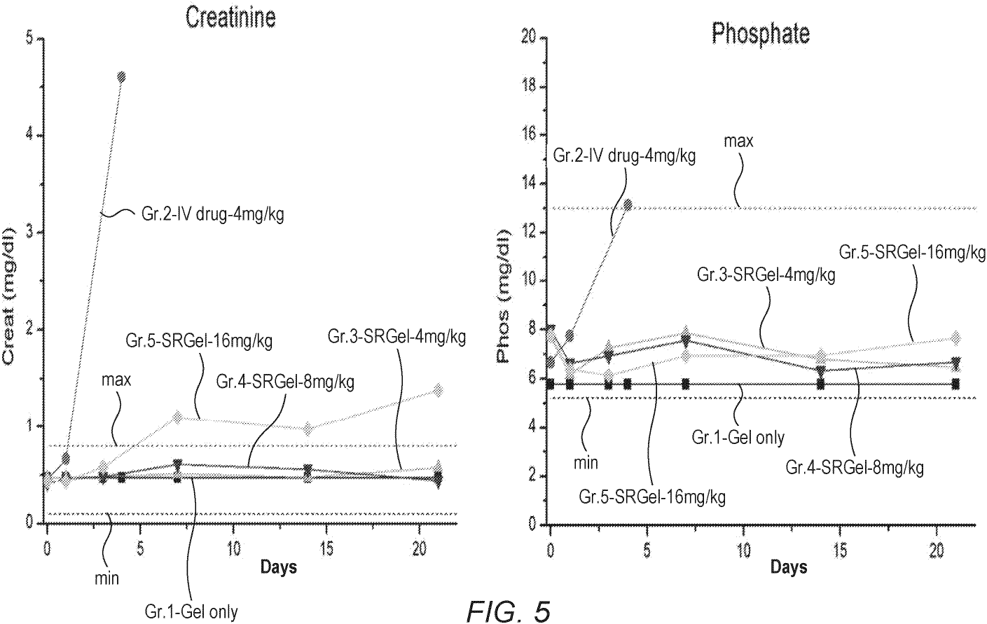

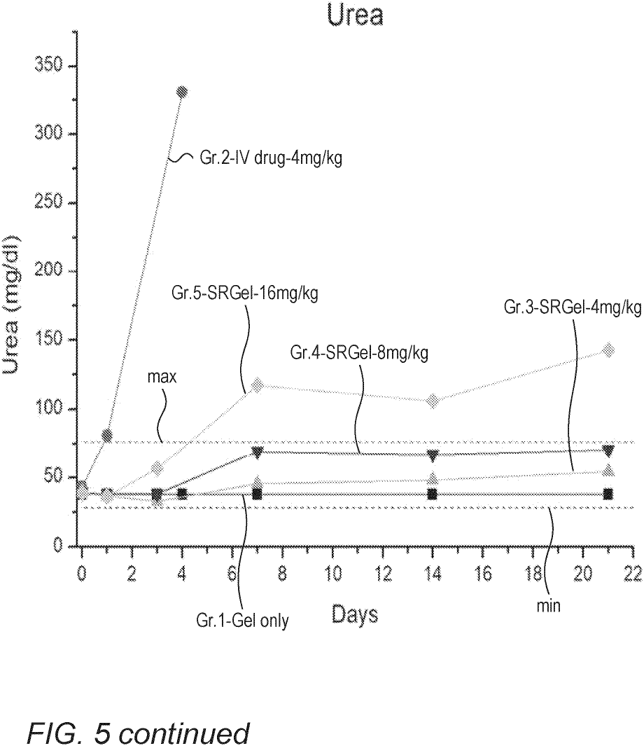

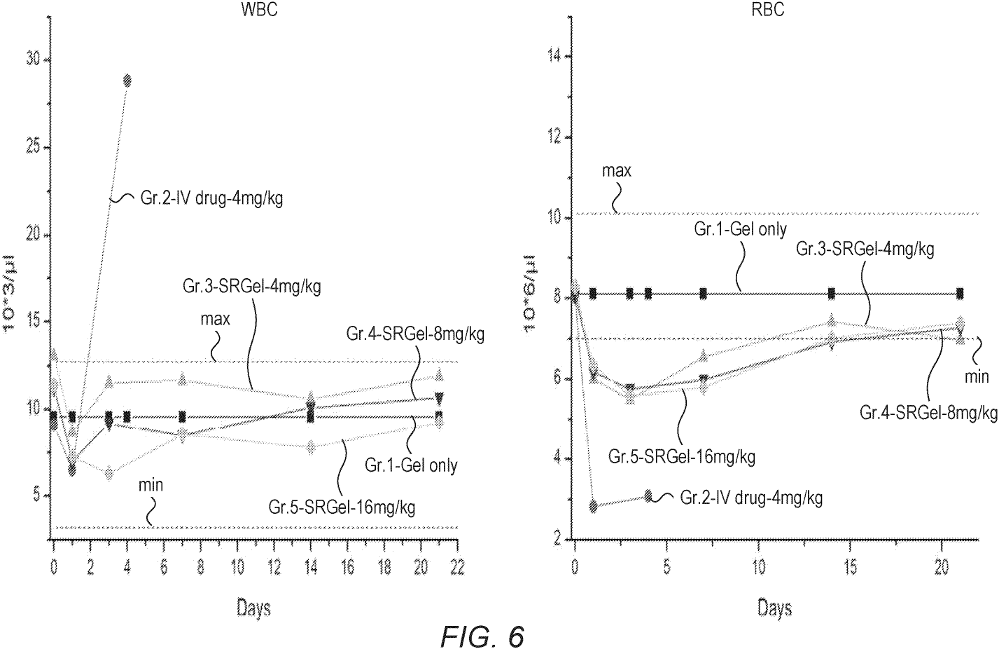

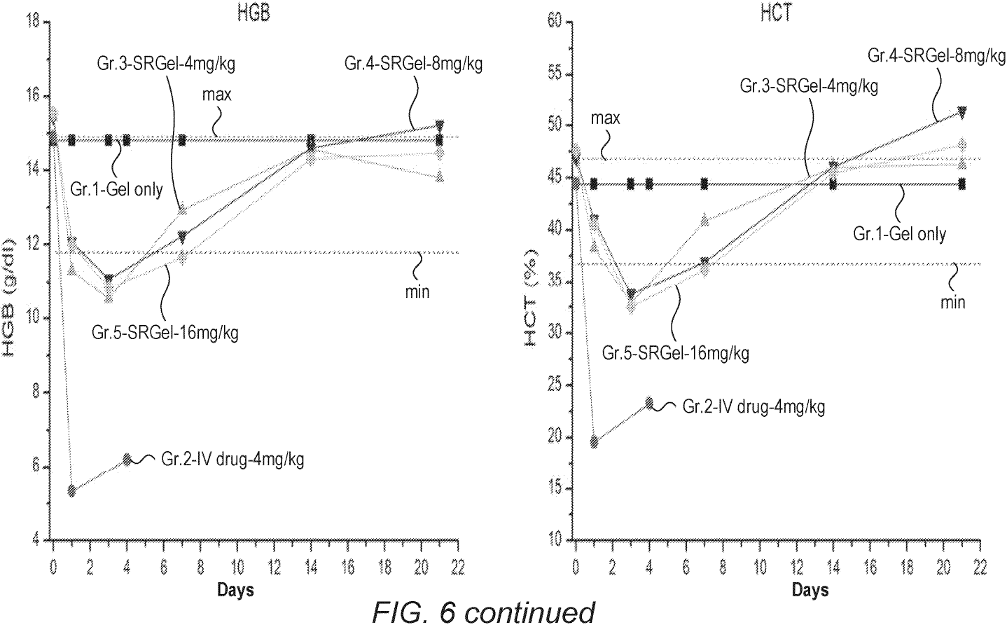

The body parameters throughout the study are given in FIG. 4. The blood analysis in given in FIG. 5.

Group 1 (SC polymer control): no toxic effects as seen in increase body weight, normal behaviour and pathology.

Group 2 (IV cisplatin solution): severe side effects on rats immediately after injection in terms of behaviour i.e. Hunched posture, Piloerection/matted fur, Signs of dehydration, Abnormal vocalization. This was translated to lower body weight, blood count and deteriorated kidneys performance by increased creatinine and urea excretion.

Groups 3-5 (SC polymer cisplatin 4, 8, 16 mg/kg): there is a clear correlation between body weight loss in the first 3 days and the release rate (amount). Although in group 5, a dose that was 4X times the amount administered to group 2 (IV) and body weight indicates similar behaviour to group 2. Still group 5 looked better than group 2 (IV) as the drug was released slowly, giving the body system time to heal itself. This can be clearly seen in the kidneys function which was slightly deviated from the norm compared to group 2.

Blood count of groups 3-5, support the hypothesis of slow release and organs recovering better using TumoCure system, as the drop in HGB, RBG & HCT was negligible compared to group 2 (IV) which showed drastic drop in markers.

### Example 10: Toxicity and Elimination of the Polymer

Poly(SA:RA)30:70 was evaluated for toxicity and elimination when administered intramuscularly and subcutaneously in rats. Doses of 100 to 300 microliter of the polymer was injected to rats and the degradation and local toxicity was determined over 3 months. No general toxicity was observed, the animals behaved normal and gained weight similar to the control group. The polymers were gradually eliminated from the site of injection over a period of 8 weeks with complete healing.

### Example 11: Release of LHRH and Somatostatin Peptides Form Polymer for Treating Cancer

Extended release of LHRH agonists are used for treating prostate cancer. In this study, LHRH, 10 mg mixture of LHRH, 2 mg and 8 mg glucose powder was incorporated in the pasty polymer Poly(SA:RA)30:70 (190 mg) by hand mixing and the formulation was placed in cap for in vitro release study. The cap loaded with the LHRH formulation was placed in 10 ml phosphate buffer pH 7.4 at 37° C. The buffer was replaced periodically for 2 weeks and the LHRH content in the releasing medium was determined by HPLC. Constant release of LHRH was observed for the two weeks period with a total of 60% of the drug content being released.

Similarly, somatostatin was incorporated in the polymer and showed a constant release for 2 weeks.

In a similar way, a protected mixture of VEGF inhibitor in amino acids, salts and sugars that protect the protein from aggregation and deterioration when in aqueous media or in dry form was employed. The powder was incorporated in the polymer paste by hand mixing at room temperature to form a uniform paste. In vitro release showed a constant release for one week.

### Example 12: Release of siRNA From Polymer

Small interfering RNAs (siRNA) technology has shown great promise as a new class of therapeutic interventions for the treatment of cancer. siRNA has been used extensively in blocking various genes and is currently being evaluated as a therapeutic for various cancers. Despite the excitement about this remarkable biological process for sequence-specific gene regulation, the major concerns limiting its use are rapid degradation by serum nuclease, poor cellular uptake, rapid renal clearance following systemic delivery, off-target effects, and induction of immune responses.

Local delivery at the tumor site should provide a solution for these drawbacks as the agent is delivered locally and not through the blood stream and it is release over days and weeks at the tumor al tissue. Naked siRNA or a mixture/complex with cationic lipids as well as buffering agents were incorporated in the polymer paste and the release study was performed as described above. A continuous release for one week was observed with little being released thereafter for the next 6 weeks.

1-59. (canceled)

60. An anticancer formulation comprising at least one anticancer agent and a carrier in a form of a polyanhydride composed of sebacic acid (SA) and ricinoleic acid (RA), the carrier having a Mw/Mn value between 1 and 2.5.

61. The formulation according to claim 60, wherein the carrier is a polyanhydride of the formula —(SA—RA)n—, wherein n is an integer between 10 and 100.

62. The formulation according to claim 60, wherein the polyanhydride is prepared by: a) melt condensation of SA and RA to form dicarboxylic acid oligomers; b) oligomer activation with acetic anhydride; c. melt polycondensation to form a polyanhydride, wherein the preparation does not comprise use of poly sebacic acid.

63. The formulation according to claim 62, wherein the oligomer activation is in the presence of a mole equivalent or less of acetic anhydride per carboxylic acid group, in the absence of a solvent.

64. The formulation according to claim 60, in a form of an implantable formulation or device or an injectable formulation.

65. The formulation according to claim 60, adapted for intratumoral delivery of said anticancer agent.

66. The formulation according to claim 60, adapted for implantation at a tumor site, following a surgical removal of a tumor or for injection into a tumor site by a needle.

67. The formulation according to claim 60, wherein the anticancer agent is a cytotoxic agent, protein or nucleotide-based biological drug for cancer treatment.

68. The formulation according to claim 60, for use in management of at least one cancer selected from blastoma, carcinoma, lymphoma, leukemia, sarcoma, mesothelioma, glioma, germinoma, choriocarcinoma, melanoma, glioblastoma, colon, head and neck, GI and lymphoid malignancies.

69. The formulation according to claim 60, for management of a solid cancer.

70. The formulation according to claim 67, wherein the solid cancer is brain cancer, breast cancer, prostate cancer, head and neck sarcoma, lung and skin cancer.

71. The formulation according to claim 67, wherein the cancer is selected from squamous cell cancer, lung cancer, small-cell lung cancer, non-small cell lung cancer, adenocarcinoma of the lung and squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and neck cancer.

72. The formulation according to claim 60, wherein the anticancer agent is selected amongst cytotoxic agents, chemotherapeutic agents, intercalating drugs, topoisomerase inhibitors, antimetabolites, antimitotic drugs, kinase inhibitors, and monoclonal antibiotics.

73. The formulation according to claim 60, wherein the anticancer agent is a platinum complex selected from carboplatin, cisplatin and oxaliplatin.

74. A method for treating or delaying or preventing progression of a cancer, the method comprising administering an effective amount of an anticancer agent in a formulation comprising a carrier in a form of a polyanhydride of the formula —(SA—RA)n—, wherein SA is sebacic acid and RA is ricinoleic acid, and wherein n is an integer between 10 and 100, the carrier having a Mw/Mn value between 1 and 2.5 or 1 and 2.

75. The method according to claim 74, wherein the polyanhydride is prepared by melt condensation of SA and RA.

76. The method according to claim 75, wherein the melt condensation is in the presence of a mole equivalent or less of acetic anhydride per carboxylic acid group, in the absence of a solvent and wherein the preparation does not comprise use of poly sebacic acid.

77. A method for treating or delaying or preventing progression of a cancer, the method comprising administering an effective amount of an anticancer agent in a formulation comprising a carrier prepared by melt condensation of SA and RA.

78. The method according to claim 77, wherein the carrier is in a form of a polyanhydride of the formula —(SA—RA)n—, wherein SA is sebacic acid and RA is ricinoleic acid, and wherein n is an integer between 10 and 100, the carrier having a Mw/Mn value between 1 and 2.5 or 1 and 2.

79. A kit comprising an anticancer drug and a carrier in a form of a polyanhydride of the formula —(SA—RA)n—, wherein SA is sebacic acid and RA is ricinoleic acid, and wherein n is an integer between 10 and 100, the carrier having a Mw/Mn value between 1 and 2.5 or 1 and 2; and instructions of use.
